(19) United States
(12) Patent Application Publication
(10) Pub. No.: US 2008/0286807 A1
(43) Pub. Date: Nov. 20, 2008

(54) Methods and Reagents for Elimination or Reduction of False Positives in the Analysis of a Sample

(75) Inventor: Antony Bakke, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: Oregon Health & Science University, Portland, OR (US)

(21) Appl. No.: 12/094,548

(22) PCT Filed: Nov. 20, 2006
(86) PCT No.: PCT/US06/61118

§ 371 Date: May 21, 2008

(30) Foreign Application Priority Data
Nov. 21, 2005 (US) 60/738792

(51) Int. Cl. G01N 33/53; G01N 33/543

(52) U.S. Cl. 435/7.2; 436/518

(57) ABSTRACT

Methods, apparatuses, and systems for eliminating or reducing false positives in assays are provided. More specifically, there is provided a method to absorb a false positive signal with an absorber in favor of detecting a true positive signal. Embodiments of the present invention include but are not limited to systems and methods for detecting antibodies with greater specificity than available assays and also for detecting a greater variety of antibodies.

### CROSS REFERENCE TO RELATED APPLICATIONS

The present application claims priority to U.S. Patent Application No. 60/738,792, filed Nov. 21, 2005, entitled “Elimination of False Positive Results in Antibody Assays,” the entire disclosure of which is hereby incorporated by reference in its entirety.

### TECHNICAL FIELD

Embodiments of the present invention relate to the field of life sciences. Embodiments of the present invention relate to reagents and methods useful for analyzing for the presence or amount of a particular analyte in a sample. Embodiments of the present invention relate to diagnosis of both autoimmune and alloimmune disorders, including, but not limited to, mechanisms to detect antiplatelet antibodies by using whole human platelets and flow cytometry.

### BACKGROUND

Patients with idiopathic (autoimmune) thrombocytopenic purpura (ITP) or newborns with alloimmune thrombocytopenia have antibodies in their blood that destroy the blood clotting cells, called platelets (e.g., antiplatelet antibodies). Without platelets, these patients can have internal bleeding and, in the case of infants, may have a risk of intracranial hemorrhage. Thus, detection of these antiplatelet antibodies is important for both diagnosis and therapy.

Immunoassays use the binding of an antibody to a specific target antigen to detect the presence of either the antibody or the antigen (e.g., the analyte to be measured). The antigen, the antibody or both are typically contained in a complex mixture, such as serum from blood, whole white blood cells, or pieces of tissue. The surface used for the measurement is called the substrate, and may be the surface of a microscope slide containing tissue, the surface of a biological cell, or the surface of a plastic well coated with purified or mixed proteins. In general, an immunoassay is designed to detect only one specific reaction, called a true positive signal, which indicates the formation of the antigen-antibody complex of interest. However, the substrate/sample may contain both the “true” antigen of interest and multiple “false” contaminating antigens. Even in assays using purified antigen, the antibody may be part of a complex protein mixture in serum. Serum contains thousands of different antibodies, including anti-antibodies (e.g., anti-immunoglobulins called Rheumatoid Factor). Since antibodies and antigens are complex structures, multiple binding reactions are possible, which may produce both “true positive” and “false positive” results. When unwanted reactions occur, the assay may be detecting a false positive signal. Many of these false positives are due to antibodies incorrectly binding to the testing substrate.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 is a simplified diagram illustrating various embodiments of the present invention.

### DETAILED DESCRIPTION OF EMBODIMENTS OF THE INVENTION

In the following detailed description, reference is made to the accompanying drawings which form a part hereof, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the description, a phrase in the form “A/B” means A or B. For the purposes of the description, a phrase in the form “A and/or B” means “(A), (B), or (A and B)”. For the purposes of the description, a phrase in the form “at least one of A, B, and C” means “(A), (B), (C), (A and B), (A and C), (B and C), or (A, B and C)”. For the purposes of the description, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

In various embodiments of the present invention, methods, apparatuses, and systems for eliminating or reducing false positives in assays are provided. In exemplary embodiments of the present invention, a computing system may be endowed with one or more components of the disclosed articles of manufacture and/or systems and may be employed to perform one or more methods as disclosed herein.

Currently utilized technologies generally either detect too many bound antibodies (no specificity, for example cannot distinguish ITP from SLE) or only detect a limited number of anti-platelet antibodies (for example, fluorescent bead assays with only a few antigens represented). Embodiments of the present invention use FRET (fluorescence resonance energy transfer), but in the reverse mode (i.e., blocking fluorescence rather than transferring the fluorescence). Therefore, in an embodiment, the term “reverse FRET” may be used to describe the fluorescence blocking/quenching relationship. In an embodiment, when a false positive signal is present, it may be absorbed by the reverse FRET absorber construct. Such a configuration increases the specificity of the method by eliminating or reducing false positives and detecting true positives.

In various embodiments of the present invention, a false positive signal in an immunoassay may be eliminated or significantly reduced by incorporating an absorber to absorb the false signal. Previous attempts have tackled such a challenge by removing the effect of an unwanted molecule from a sample (for example, by competitively binding) to avoid the unwanted binding of that molecule to the target of interest. See, for example, US Patent Application 2004/0018556 to Cantor. However, in embodiments of the present invention, there is provided a mechanism to address the binding of a molecule to an unwanted target. Such embodiments address the concerns of non-specific binding of a molecule to a secondary/unwanted site resulting in a false positive signal.

Thus, in an embodiment there is provided a method of measuring a positive receptor target in a sample, comprising providing a sample having a substrate, the substrate having a positive receptor target including at least one binding site and a false positive receptor including at least two binding sites; contacting the sample with a fluorescent probe adapted to bind to the at least one binding site of the positive receptor target and nonspecifically to at least one of the at least two binding sites of the false positive receptor, wherein when the fluorescent probe is bound to a binding site of the positive receptor target, the fluorescent probe fluoresces; and contacting the sample with an absorber construct adapted to bind to at least one of the at least two binding sites of the false positive receptor, wherein when the absorber construct is bound to a binding site of the false positive receptor and when the fluorescent probe is bound to an adjacent binding site on the false positive receptor, the fluorescence of the fluorescent probe is absorbed, at least partially, by the absorber construct.

In an embodiment, for example, two separate antibody systems may be employed in combination. A signal antibody system may be adapted to bind to a target antigen and generate a fluorescent signal and a second antibody system may be adapted to eliminate false-positive signals. In such an embodiment, the two antibody systems may simultaneously bind to adjacent sites on a substrate without cross-reacting.

In an embodiment of the present invention, the signal antibody system may comprise a non-fluorescent primary antibody and a fluorescent secondary antibody. For example, in an embodiment, the non-fluorescent primary antibody may be human immunoglobulin G (“IgG”) and the fluorescent secondary antibody may be a fluorescent-labeled anti-human immunoglobulin that binds to human IgG.

In an embodiment of the present invention, the second antibody system may comprise a second primary antibody and an absorber molecule. For example, the second primary antibody may be a mouse monoclonal antibody and the absorber antibody molecule may be an anti-mouse IgG antibody.

In an embodiment, an absorber may be adapted to quench a fluorescent signal given off by the signal antibody system's fluorescent secondary antibody. In an embodiment of the present invention, the fluorescent signal may be quenched by the absorber using Fluorescence Resonance Energy Transfer or Förster Resonance Energy Transfer (“FRET”). In embodiments, all or some of the fluorescence emitted by a fluorescent molecule may be absorbed by an absorber, such as approximately 30%, 50%, 70%, 90% or more.

In embodiments of the present invention, an absorber may be fluorescent or non-fluorescent. In an embodiment, fluorescent conjugates may be, for example, those that absorb FITC fluorescence (emission between 505-550 nm), including Texas Red (excitation/absorption between 570-600 nm), rhodamine (excitation/absorption from 530-560 nm), cyanine 3 (excitation/absorption from 525-570 nm), cyanine 5 (excitation/absorption from 560-635 nm), etc. In an embodiment, non-fluorescent absorbers may also be used, which offer the added benefit of not creating fluorescence that may confuse the results. An exemplary absorbing molecule QSY7 (available from Invitrogen/Molecular Probes, Eugene, Oreg.; available in a reactive form—succinimidyl ester—that covalently reacts with amino groups on the antibody molecule). In an embodiment, a non-fluorescent absorber may be conjugated, for example, to an anti-mouse IgG to form an absorbing conjugate.

In an embodiment, an assay may be enhanced by increasing the fluorescence absorption, for example by incorporating a biotin-avidin system or a tertiary antibody system. In embodiments, such systems allow for the conjugation of additional fluorescence absorbing side chains, thus increasing absorption capacity. In addition, in an embodiment, a false positive receptor may have more than two binding sites and thus a system may be adapted to allow binding of more than one absorber construct. In an embodiment with multiple absorber constructs on a single false positive receptor, the multiple absorber constructs may differ from each other and may be adapted to bind to different binding sites on the receptor.

In an embodiment, there is provided a two-color flow cytometry assay. In an embodiment, the signal to noise ratio of the assay may be optimized with fluorescent beads (such as available from Spherotech, Libertyville, Ill.). In an embodiment, individual fluorochromes may produce signals in other fluorescent channels. Thus, in an embodiment, these signals may be eliminated or reduced with proper compensation of the flow cytometer. In an embodiment, compensation controls for two color flow cytometry includes beads (such as CompBeads, BD Biosciences) and cells (platelets) labeled with single fluorochromes.

In a further embodiment of the present invention, a primary antibody may bind to a false positive site on a substrate and a secondary antibody may be adapted to bind at a site adjacent to the primary false positive site.

As mentioned above, in an embodiment, the secondary antibody may be adapted to bind at a site adjacent to the primary false positive site. For the purposes of the present invention, the term “adjacent” refers to a sufficient proximity between the molecules of interest or binding sites such that when the multiple binding events occur, the absorber is sufficiently close to the fluorescent molecule to absorb the fluorescence, at least partially. The term “adjacent” also refers to binding sites that are next to each other on a molecule or receptor. In an embodiment, two molecules that are adjacent to each other may be sufficiently close such that there are no intervening molecules that would block the absorption by the absorbing molecule of the fluorescence emitted from the fluorescent molecule.

Current immunoassay methods make use of 2 monoclonal antibodies binding to 1 molecule for detection and reduction of false positives. This may be adequate when the analyte to be detected is a well understood molecule such as a single protein or smaller antigen. However, when the analyte is not well characterized or is part of a complex mixture, the current methods are often inadequate. In one embodiment of the invention, platelets containing many different proteins are a complex target and autoantibodies produced in the patient are the analyte to be measured. In such an embodiment, the exact target on the platelet may be unknown. Thus, embodiments of the present invention make use of 2 monoclonal antibodies to identify 2 adjacent molecules on a platelet. Such a method provides information on the analyte of interest and on confounding, non-specific reactions. The occurrence of nonspecific reactions may be attributed to substances (interferents including, e.g., immune complexes) present in a test sample, which may become bound to the complex target (e.g. platelet) and test reagents. Generally, for many immunoassays, these interfering factors bind to the complex target at a different site than the analyte being detected, but still lead to the formation of positively detected complexes even in the absence of the analyte of interest (such as an autoantibody).

Thus, in an embodiment of the present invention, a method is provided to detect positive signals and eliminate false positive signals. In an embodiment, the second adjacent reaction allows for the identification or reduction/elimination of false positives. In such an example, false positives may be due to immune complexes binding to Fc receptors on the platelet. In an embodiment, adjacent binding of a monoclonal antibody to a site on the Fc receptor separate from the immune complex binding site allows elimination or reduction of the false positive signal due to the immune complex.

In an embodiment of the present invention, a flow cytometry FRET assay may be used to analyze and characterize the binding and fluorescence of the various molecules/components.

In an embodiment, whole blood, or a component of fractionated whole blood, may be analyzed using a device, such as a flow cytometer, to measure the analyte of interest or to identify the presence of a target of interest. Prior methods generally rely on the use of plasma or serum derived from whole blood. However, fractionation of the whole blood may lead to loss of the analyte of interest (e.g. autoantibody) and resulting false negatives. Thus, in an embodiment, the use of whole blood may be preferred.

Previous immunoassay methods make use of serum binding to a solid phase, e.g. plastic enzyme immunoassay plate, and two monoclonal antibodies to identify the analyte. Such a method uses a simple, well characterized target and solid phase measurement. Previous methods rely on only a few protein targets and many targets are not fully characterized. Unknown targets cannot be detected and represent false negatives. In an embodiment of the present invention, whole cells, such as platelets may be used, since there are many more targets present on the surface of cells. Such a use of a complex target reduces false negatives.

In an embodiment, the use of the fluid phase and a flow cytometer allows separation of complex populations of cells in blood without extensive, time consuming, prior purification of one cell or one target. The flow cytometer acts like a rapid, continuous flow microscope for detecting both the cell type and any bound reactants.

Immune complexes in a patient's serum may lead to serious consequences, such as interacting with the cell (platelet) used in the assay. This interaction may lead to assay interferences which may produce false positives. In addition, some immune complexes contain rheumatoid factors (IgM anti-IgG antibodies) which may cause false negatives. Either result may lead to an incorrect diagnosis. Endogenous human heterophilic antibodies that have the ability to bind to immunoglobulins (i.e. rheumatoid factors) of other species may be present in serum or plasma of 10% or more of patients.

Immune complexes and/or heterophilic antibodies may develop resulting from different exposures, such as rheumatoid arthritis, vaccinations, influenza, animal contact (pets), allergies, special diets, (e.g., cheese), blood transfusions, contact therapy, autoimmune diseases, dialysis, patent medicines, maternal transfer, cardiac myopathy, and G.I. disease (E. coli).

In an embodiment of the present invention, there is provided a kit, comprising a fluorescent probe adapted to bind to at least one binding site of a positive receptor target and nonspecifically to at least one of at least two binding sites of a false positive receptor; and an absorber construct adapted to bind to at least one of the at least two binding sites of the false positive receptor, wherein the absorber construct is adapted to absorb fluorescence, at least partially, from the fluorescent probe when the fluorescent probe and the absorber construct are bound to adjacent binding sites on the false positive receptor.

In an exemplary embodiment of the present invention, serum from the blood of a patient, normal human platelets, monoclonal antibodies against specific platelets' surface proteins (CD antigens), fluorescent anti-human immunoglobulin and anti-mouse IgG with a fluorescence absorber may be used in a flow cytometry system to detect the presence of antibody bound to receptors/targets on the platelet surface and to eliminate false positive results based on adjacent binding of an absorber antibody. A true positive result for this assay may occur when the human serum antibody is bound to a platelet surface protein and a positive fluorescent signal is emitted and detected. A false positive may occur when the human serum antibody is bound non-specifically to a surface protein and the monoclonal antibody is bound to the same surface protein at a different binding site. In an embodiment of the present invention, with false positive binding, a fluorescence signal may be absent (quenched) due to absorption by an adjacent molecule such as the adjacent anti-mouse IgG with fluorescence absorber. In embodiments, such an assay may detect the presence of antibody bound to the platelet (or other substrate) surface, specificity for the specific surface protein, and orientation of the antibody binding.

In an exemplary embodiment of the present invention, sera from patients with immune thrombocytopenia, systemic lupus erythematosus, normal controls, and heat aggregated normal control sera may be tested as follows:
1. Anticoaggulated blood may be collected from normal human controls and platelets may be isolated by centrifugation. The platelets may be counted and their concentration may be adjusted to a desired value. For example, the platelet concentration may be adjusted to 250,000 per microliter.
2. Dilutions of human sera may be added to a quantity of normal platelets along with murine monoclonal antibodies to CD32 (Fc receptor) and CD42b (von Willebrand receptor) and incubated. In an embodiment of the present invention, the quantity of normal platelets may be approximately 10 microliters. In a further embodiment of the present invention, the dilutions of human sera, the quantity of normal platelets, and the murine monoclonal antibodies may be incubated for 30 minutes. The CD42b antibody may be conjugated to a separate fluorochrome, for example phycoerythrin, to identify platelets.
3. After incubation, the platelets may be washed twice by centrifugation with phosphate buffered saline containing 0.1% bovine serum albumin and 0.1% sodium azide to remove unbound antibodies.
4. Fluorescent anti-human IgG (goat anti-human IgG conjugated to FITC) and absorbing anti-murine IgG (goat anti-mouse IgG conjugated to Texas Red or QSY 9) may be added and incubated. In an embodiment of the present invention, the fluorescent anti-human IgG, absorbing anti-murine IgG dilutions of human sera, and platelet mixture may be incubated for 30 minutes.
5. After the second incubation, the platelets may be washed twice by centrifugation with phosphate buffered saline containing 0.1% bovine serum albumin and 0.1% sodium azide to remove unbound antibodies.
6. Labeled platelets may be analyzed on a flow cytometry instrument.
7. The data may be analyzed for percent positive staining of platelets and groups may be compared by analysis of variance (ANOVA). In various embodiments of the present invention, sensitivity and specificity may be calculated and are expected to be greater than 90 percent for both.

While the above example examined conditions such as immune thrombocytopenia and systemic lupus erythematosus, other diseases and conditions may be examined in accordance with the teachings of embodiments of the present invention. The mechanisms of providing a quenching relationship between a non-specifically binding molecule having a fluorescent tag to an unwanted binding site and a nearby or adjacent construct having a fluorescent absorber to reduce or eliminate false positives may be extended to a variety of molecules, antibodies, etc. in accordance with the teachings herein.

In an embodiment of the present invention, there is provided a method to restrict an antibody label from emitting a false positive signal comprising providing the antibody label and an absorber, wherein a transfer of energy from the antibody label to the absorber occurs when the antibody label and absorber are adjacent to one another, and such transfer of energy restricts or absorbs an emission of radiation from the antibody label. In a further embodiment of the present invention, said transferring energy from an antibody label to the adjacent absorber comprises FRET. In a further embodiment of the present invention, the antibody label comprises a fluorescent label.

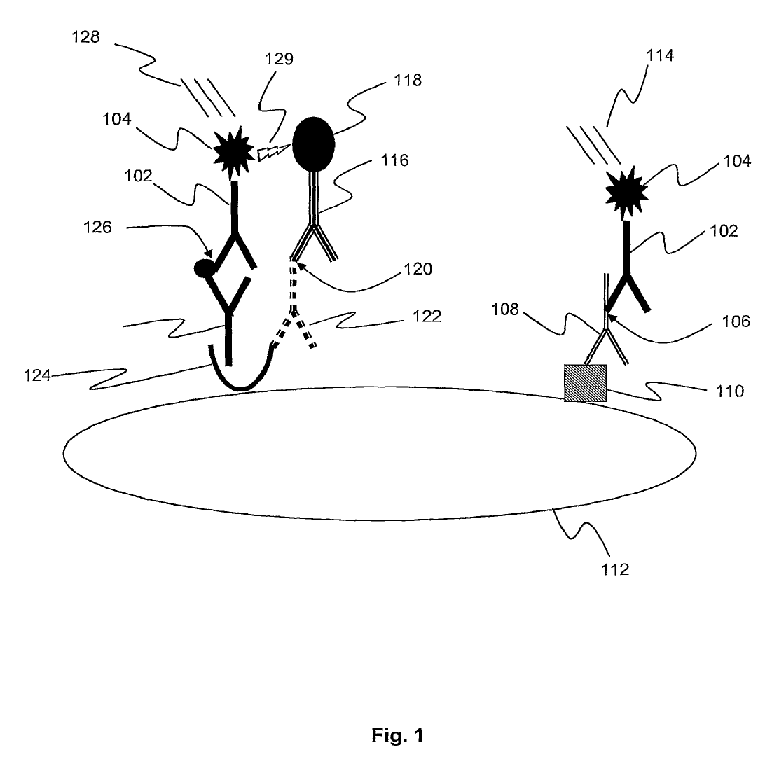

Referring now to FIG. 1, wherein a simplified diagram illustrating various embodiments of the present invention is shown. As illustrated, a signal antibody 102 may be coupled to a label 104. Further, signal antibody 102 may be employed to bind to a target binding site 106. In various embodiments of the present invention, signal antibody 102 may be an antiantibody and target binding site 106 may be located on a target antibody 108. More specifically, signal antibody 102 may be an anti-human immunoglobulin adapted to bind to human IgG. In an embodiment of the present invention, target antibody 108 may be an immunoglobulin, such as IgG. In another embodiment of the present invention, target antibody 108 may be an autoantibody for a patient with either idiopathic thrombocytopenic purpura or alloimmune thrombocytopenia.

In an embodiment of the present invention, target antibody 108 may bind to a target 110 located on substrate 112. In various embodiments of the present invention, target 110 may be an antigen and substrate 112 may be a cell. In particular, target 110 may be a platelet antigen and substrate 112 may be a platelet.

In various embodiments, label 104 may emit radiation. In an embodiment of the invention, label 104 may be a fluorescent label and the radiation may be fluorescent radiation. For example, label 104 may be fluorescein isothiocyanate (FITC) or phycoerythrin (PE). In an embodiment of the present invention, when signal antibody 102 is bound to target binding site 106, label 104 may emit true positive signal 114. Thus, in an embodiment of the present invention, by detecting true positive signal 114, the presence of a specific autoantibody or alloantibody may be detected.

According to various embodiments of the present invention, an absorber antibody 116 may be coupled to an absorber 118. In exemplary embodiments of the present invention, the absorber 118 may be a dye, as is known in the art, such as Texas Red or QSY 9. Absorber antibody 116 may be employed to bind an absorber binding site 120. In various embodiments of the present invention, absorber antibody 116 may be an immunoglobulin and absorber binding site 120 may be located on a primary antibody 122. In an embodiment, primary antibody 122 may be a monoclonal antibody. In particular, primary antibody 122 may be a mouse anti-human Fc receptor monoclonal antibody. In a further embodiment, absorber antibody 116 may be an anti-mouse IgG adapted to bind to primary antibody 122. In an embodiment of the present invention, primary antibody 122 may bind to a contaminating antigen 124 located on substrate 112. In exemplary embodiments of the present invention, contaminating antigen 124 may be a glycoprotein, such as an Fc receptor. In various embodiments, signal antibody 102 may bind to a contaminating site 126. Contaminating site 126 may, for example, be located on an immune complex 127, which may be bound to a contaminating antigen 124. In an embodiment of the present invention, when signal antibody 102 is bound to contaminating site 126, label 104 may emit false positive signal 128.

In various embodiments, energy 129 produced by label 104 may be transferred to absorber 118 when absorber 118 is adjacent to label 104. In an exemplary embodiment of the present invention, absorber 118 may be adjacent to label 104 when an intermolecular distance between label 104 and absorber 118 is in a range of about 1 to 200 Angstroms. In various embodiments of the present invention absorber 118 may be adjacent to label 104 when signal antibody 102 is bound to contaminating site 126 and absorber antibody 116 is bound to absorber binding site 120.

In an embodiment of the present invention, energy 129 may be transferred to absorber 118 by FRET. In such an embodiment, the intermolecular distance between label 104 and absorber 118 may, for example, be in a range of about 10 to 100 Angstroms.

In an embodiment of the present invention, the transfer of energy 129 from label 104 to absorber 118 may quench label 104. In such an embodiment, quenching label 104 may restrict label 104 from emitting false positive signal 128. Resultantly, absorber 118 may restrict label 104 from emitting false positive signal 128 when absorber 118 is adjacent to label 104. Thus, according to various embodiments of the present invention, a method is provided to reduce or block false positive signal 128. In exemplary embodiments of the present invention, absorber 118 may be a dye, as is known in the art, such as Texas Red or QSY 9.

While FIG. 1 is shown with various constructs (such as, in an embodiment, an immune complex 127 and an antibody 102), in embodiments, a probe may directly bind to a target, antigen, or receptor without the formation of a construct or without using a linking molecule.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein.

What is claimed is:

1. A method of measuring a positive receptor target in a sample, comprising:
providing a sample having a substrate, said substrate having a positive receptor target including at least one binding site and a false positive receptor including at least two binding sites;
contacting the sample with a fluorescent probe adapted to bind to said at least one binding site of said positive receptor target and nonspecifically to at least one of said at least two binding sites of said false positive receptor, wherein when said fluorescent probe is bound to a binding site of said positive receptor target, said fluorescent probe fluoresces; and
contacting the sample with an absorber construct adapted to bind to at least one of said at least two binding sites of said false positive receptor, wherein when said absorber construct is bound to a binding site of said false positive receptor and when said fluorescent probe is bound to an adjacent binding site on said false positive receptor, the fluorescence of said fluorescent probe is absorbed, at least partially, by said absorber construct.

2. The method of claim 1, further comprising measuring the fluorescence of the bound fluorescent probe using a flow cytometer.

3. The method of claim 1, wherein said substrate is a cell or platelet.

4. The method of claim 1, wherein said positive receptor target is a cell- or platelet-surface antigen.

5. The method of claim 1, wherein said positive receptor target is an antibody or autoantibody bound to a surface receptor on said substrate.

6. The method of claim 1, wherein said positive receptor target is IgG bound to a surface receptor on said substrate.

7. The method of claim 1, wherein said sample is a whole blood sample.

8. The method of claim 1, wherein at least 50% of the fluorescence from said fluorescent probe bound to the adjacent binding site on said false positive receptor is absorbed by the absorber construct.

9. The method of claim 1, wherein said fluorescent probe comprises a fluorescently conjugated anti-human IgG.

10. The method of claim 9, wherein said fluorescently conjugated anti-human IgG is fluorescently labeled with fluorescein isothiocyanate or phycoerythrin.

11. The method of claim 1, wherein said fluorescent probe is a fluorescently labeled antibody.

12. The method of claim 1, wherein said fluorescent probe is a fluorescently labeled antiantibody.

13. The method of claim 1, wherein said absorber construct comprises an absorber molecule and a linker molecule adapted to bind to at least one of said at least two binding sites of said false positive receptor.

14. The method of claim 13, wherein said absorber molecule comprises a fluorescent conjugate.

15. The method of claim 14, wherein said fluorescent conjugate comprises Texas Red, rhodamine, cyanine 3, or cyanine 5.

16. The method of claim 13, wherein said linker molecule comprises an antibody.

17. The method of claim 13, wherein said linker molecule comprises an anti-mouse IgG.

18. The method of claim 13, wherein said linker molecule comprises a chain of linker molecules comprising an anti-mouse IgG bound to said absorber molecule and a mouse monoclonal antibody having an anti-human Fc receptor.

19. A kit, comprising:
a fluorescent probe adapted to bind to at least one binding site of a positive receptor target and nonspecifically to at least one of at least two binding sites of a false positive receptor; and
an absorber construct adapted to bind to at least one of said at least two binding sites of said false positive receptor, wherein said absorber construct is adapted to absorb fluorescence, at least partially, from said fluorescent probe when said fluorescent probe and said absorber construct are bound to adjacent binding sites on said false positive receptor.

20. The kit of claim 19, wherein said fluorescent probe is a fluorescently labeled antibody.

21. The kit of claim 19, wherein said fluorescent probe is a fluorescently labeled antiantibody.

22. The kit of claim 19, wherein said fluorescent probe comprises a fluorescently conjugated anti-human IgG.

23. The kit of claim 22, wherein said fluorescently conjugated anti-human IgG is fluorescently labeled with fluorescein isothiocyanate or phycoerythrin.

24. The kit of claim 19, wherein said absorber construct comprises an absorber molecule and a linker molecule adapted to bind to at least one of said at least two binding sites of said false positive receptor.

25. The kit of claim 24, wherein said absorber molecule comprises a fluorescent conjugate.

26. The kit of claim 25, wherein said fluorescent conjugate comprises Texas Red, rhodamine, cyanine 3, or cyanine 5.

27. The method of claim 24, wherein said linker molecule comprises an antibody.

28. The kit of claim 24, wherein said linker molecule comprises an anti-mouse IgG.

29. The kit of claim 24, wherein said linker molecule comprises a chain of linker molecules comprising an anti-mouse IgG bound to said absorber molecule and a mouse monoclonal antibody having an anti-human Fc receptor.

30. The kit of claim 29, wherein said fluorescent probe comprises a fluorescently conjugated anti-human IgG.

31. The kit of claim 30, wherein said fluorescently conjugated anti-human IgG is fluorescently labeled with fluorescein isothiocyanate or phycoerythrin.
